(19) United States
(12) Patent Application Publication
(10) Pub. No.: US 2002/0187484 A1
(43) Pub. Date: Dec. 12, 2002

(54) Molecular break lights probes for detecting nucleotide cleavage

(76) Inventors: Jon Thorson, Madison, WI (US); James Prudent, Madison, WI (US)

Correspondence Address: Deborah A. Somerville, KENYON & KENYON, One Broadway, New York, NY, 10004, US

(21) Appl. No.: 09/993,757

(22) Filed: Nov. 27, 2001

Related U.S. Application Data
(60) Provisional application No. 60/253,382, filed on Nov. 27, 2000.

(51) Int. Cl. C12Q 1/68; C12P 19/34; C12N 9/22

(52) U.S. Cl. 435/6; 435/91.2; 435/199

(57) ABSTRACT

Modified hairpin-forming oligonucleotide to continuously assess nucleotide cleavage by enediynes and other nucleic acid cleavage agents are provided. These oligonucleotide probes, which are also referred to herein as “molecular break lights,” are also useful for continuous assessment of protection of nucleotides from cleavage agents. Probes according to the present invention are useful in assays; improved assays, including multiplexed assays, utilizing such pairs of molecules or moieties; and assay kits that include such pairs. Methods of using the probes are also provided.

### GOVERNMENT SUPPORT

The invention described herein was made with assistance of a NIH grant CA84374. The Government may have certain rights in the invention.

### BACKGROUND

1. Field of the Invention

This invention relates to nucleic acid cleavage probes containing fluorophore and quencher, and kits and assays containing and employing them.

2. Background

Fluorescence resonance energy transfer, or “FRET”, assays have been used for many purposes. In FRET assays, a change in fluorescence is caused by a change in the distance separating a first fluorophore from an interacting resonance energy acceptor, either another fluorophore or a quencher. Combinations of a fluorophore and an interacting molecule or moiety, including quenching molecules or moieties, are known as “FRET pairs.” The mechanism of FRET-pair interaction requires that the absorption spectrum of one member of the pair overlaps the emission spectrum of the other member, the first fluorophore. If the interacting molecule or moiety is a quencher, its absorption spectrum must overlap the emission spectrum of the fluorophore. Stryer, L., “Fluorescence Energy Transfer as a Spectroscopic Ruler,” Ann. Rev. Biochem. 1978, 47: 819-846 (“Stryer, L. 1978”); Biophysical Chemistry part II, Techniques for the Study of Biological Structure and Function, C. R. Cantor and P. R. Schimmel, pages 448-455 (W. H. Freeman and Co., San Francisco, U.S.A., 1980) (“Cantor and Schimmel 1980”), and Selvin, P. R., “Fluorescence Resonance Energy Transfer,” Methods in Enzymology 246: 300-335 (1995) (“Selvin, P. R. 1995”).

One suitable FRET pair disclosed in Matayoshi et al. 1990, Science 247: 954-958, includes DABCYL as a quenching moiety (or quenching label) and EDANS as a fluorophore (or fluorescent label). A variety of labeled nucleic acid hybridization probes and detection assays that utilize FRET and FRET pairs are known. One such scheme is described by Cardullo et al., Proc. Natl. Acad. Sci. U.S.A. 85: 8790-8794 (1988) and in Heller et al. EP 0 070 685 A2. The scheme described in Cardullo and Heller uses a probe comprising a pair of oligodeoxynucleotides complementary to contiguous regions of a target DNA strand. One probe molecule contains a fluorescent label, a fluorophore, on its 5′ end, and the other probe molecule contains a different fluorescent label, also a fluorophore, on its 3′ end. When the probe is hybridized to the target sequence, the two labels are brought very close to each other. When the sample is stimulated by light of an appropriate frequency, fluorescence resonance energy transfer from one label to the other occurs. FRET produces a measurable change in spectral response from the labels, signaling the presence of targets. One label could be a “quencher,” which in this application is meant an interactive moiety (or molecule) that releases the accepted energy as heat.

Another solution-phase scheme utilizes a probe comprising a pair of oligodeoxynucleotides and a FRET pair. However, in that scheme, the two probe molecules are completely complementary both to each other and to complementary strands of a target DNA. Morrison and Stols, “Sensitive Fluorescence-Based Thermodynamic and Kinetic Measurements of DNA Hybridization in Solution,” Biochemistry 32: 309-3104 (1993) and Morrison EP 0 232 967 A2. Each probe molecule includes a fluorophore conjugated to its 3′ end and a quenching moiety conjugated to its 5′ end. When the two oligonucleotide probe molecules are annealed to each other, the fluorophore of each is held in close proximity to the quenching moiety of the other. With the probe in this conformation, if the fluorophore is then stimulated by light of an appropriate wavelength, the fluorescence is quenched by the quenching moiety. However, when either probe molecule is bound to a target, the quenching effect of the complementary probe molecule is absent. In this conformation a signal is generated. The probe molecules are too long to self-quench by FRET when in the target-bound conformation.

A solution-phase scheme that utilizes FRET pairs and the phenomenon known as strand displacement is described by Diamond et al. U.S. Pat. No. 4,766,062; Collins et al. U.S. Pat. No. 4,752,566; Fritsch et al. U.S. Pat. Nos. 4,725,536 and 4,725,537. Typically, these assays involve a probe comprising a bimolecular nucleic acid complex. A shorter single strand comprising a subset of the target sequence is annealed to a longer single strand which comprises the entire target binding region of the probe. The probe in this configuration thus comprises both single-stranded and double-stranded portions. Diamond et al. proposed that these probes may further comprise either a ³²P label attached to the shorter strand or a fluorophore and a quencher moiety which could be held in proximity to each other when the probe conformation is that complex.

Another type of molecular probe assay utilizing a FRET pair is described in European Patent Application 0 601 889 A3, publication date Jun. 15, 1994.

Another type of nucleic acid hybridization probe assay utilizing a FRET pair is the TaqMan® assay described in Gelfand et al. U.S. Pat. No. 5,210,015, and Livak et al. U.S. Pat. No. 5,538,848. The probe is a single-stranded oligonucleotide labeled with a FRET pair. In a TaqMan® assay, a DNA polymerase releases single or multiple nucleotides by cleavage of the oligonucleotide probe when it is hybridized to a target strand. That release provides a way to separate the quencher label and the fluorophore label of the FRET pair. According to Livak et al. “straightening” of an end-labeled TaqMan® probe also reduces quenching.

Yet another type of nucleic acid hybridization probe assay utilizing FRET pairs is described in Tyagi et al. now U.S. Pat. No. 5,925,517 and PCT Application No. WO 95/13399, which utilizes labeled oligonucleotide probes, which are often referred to as “Molecular Beacons.” Tyagi, S. and Kramer, F. R., “Molecular Beacons: Probes that Fluoresce upon Hybridization,” Nature Biotechnology 14: 303-308 (1996). A molecular beacon probe is an oligonucleotide whose end regions hybridize with one another in the absence of target but are separated if the central portion of the probe hybridizes to its target sequence. The rigidity of the probe-target hybrid precludes the simultaneous existence of both the probe-target hybrid and the intramolecular hybrid formed by the end regions. Consequently, the probe undergoes a conformational change in which the smaller hybrid formed by the end regions disassociates, and the end regions are separated from each other by the rigid probe-target hybrid.

However, with the exception of assays for DNAse, continuous assays for most enzymatic and small molecule-catalyzed DNA cleavage events were unavailable prior to the work of the present inventors. “Molecular beacon” assays are useful only for PCR applications and for studying DNA and RNA hybridization.

There have previously been a few reports of the application of FRET to assay enzymatic cleavage using a fluorescent-modified oligonucleotide/unlabeled oligonucleotide complement pair. However, these techniques have many limitations. For example, significant background fluorescence, as a result of poor fluorescence-quenching by the hybridizing strand, is often a problem.

Calicheamicin γ₁^{I} (FIG. 1A) from Micromonospora echinospora spp. calichensis is over 1000 times more potent than adriamycin, clinically one of the most useful antitumor agents available. A prominent member of the enediyne family, calicheamicin is a premiere example of nature's ingenuity. See, e.g., Thorson, J. S. et al. Curr. Pharmaceutical Design 6(18):1841-79 (2000); Thorson, J. S. et al Bioorgan. Chem. 27: 172-188 (1999); Borders, D. B. et al. in Enediyne Antibiotics as AntitumorAgents, Marcel Dekker, New York, N.Y. (1995); Smith, A. L. et al. J. Med. Chem. 39: 2103-2117 (1996); Nicolaou, K. C. et al. Proc. Natl.Acad. Sci. USA 90: 5881-5888 (1993); Nicolaou, K. C. et al. Angew. Chem. Intl. Ed. 30: 1387-1416 (1991).

Of the two distinct structural regions within calicheamicin, the aryltetrasaccharide is comprised of a unique set of carbohydrate and aromatic units which site-specifically deliver the metabolite into the minor groove of DNA; while the aglycone, or “warhead”, consists of a highly functionalized bicyclo[7.3.1]tridecadiynene core structure with an allylic trisulfide serving as the triggering mechanism. See, e.g., 7. Zein, N., et al. Science 244: 697-699 (1989); Zein, N., et al. Science 240: 1198-1201 (1988); Kumar, R. A., et al., J. Mol. Biol. 265: 187-201 (1997).

Aromatization of the bicyclo[7,3.1]tridecadiynene core structure, via a 1,4-dehydrobenzene-diradical results in the site specific oxidative double strand scission of the targeted DNA and this extraordinary reactivity has sparked considerable interest in the pharmaceutical industry. See, e.g., Sievers, E. L., et al. Blood 93: 3678-3684 (1999); Bemstein, I. D. Leukemia 14: 474-475 (2000).

While extensive effort has been applied to understanding the mechanism by which enediynes cleave nucleic acids, a continuous assay for this phenomenon is still lacking. In fact, with the exception of assays for DNAse, continuous assays for most enzymatic and small molecule-catalyzed nucleic acid cleavage events are unavailable. The effort to understand calicheamicin biosynthesis, self-resistance and mode of action is just one example of the research that would be facilitated by continuous assays for most enzymatic and small molecule-catalyzed nucleic acid cleavage events.

### SUMMARY OF THE INVENTION

Previous assays for enediyne cleavage of nucleotides relied upon discontinuous assays using radioactive nucleotide probes, electrophoresis and subsequent phosphoimager analysis. In contrast, by using methods and reagents (molecular break lights) of the present invention, one can directly follow the extent of nucleotide cleavage by a specific nucleic acid cleavage agent (also called a “nucleotide cleavage agent”), such as an enediyne in real time with high sensitivity and low background.

The present invention provides a modified hairpin-forming oligonucleotide to continuously assess nucleotide cleavage by enediynes and other nucleic acid cleavage agents. These oligonucleotide probes, which are also referred to herein as “molecular break lights, are also useful for continuous assessment of protection of nucleotides from cleavage agents.

Probes according to the present invention are useful in assays; improved assays, including multiplexed assays, utilizing such pairs of molecules or moieties; and assay kits that include such pairs.

The present invention provides processes for evaluating activity of nucleic acid cleavage agents present in a sample. In certain embodiments, the processes comprise: a. incubating the sample with a probe, the probe comprising: an oligonucleotide that forms a stem loop structure, a fluorophore, and a quencher, wherein the fluorophore and the quencher are positioned such that the fluorophore fluoresces less when the probe is intact than when the probe is cleaved; b. measuring the level of fluorescence of the probe; and c. correlating amount of fluorescence with activity of the nucleic acid cleavage agent.

The present invention also provides processes for detecting the presence of a nucleic acid cleavage agent in a sample. In certain embodiments, the processes comprise: incubating the sample with a probe, the probe comprising an oligonucleotide that forms a stem loop structure, a fluorophore, and a quencher, wherein the fluorophore and the quencher are positioned such that the fluorophore fluoresces less when the probe is intact than when the probe is cleaved; and b. measuring the level of fluorescence of the probe.

The nucleic acid cleavage agent may be, e.g., an enzyme, such as a nuclease. Examples of nucleases the activity or presence of which may be assayed using the processes and probes of the present invention include exonucleases and endonucleases, such as restriction endonucleases. Other examples of nucleic acid cleavage agents the activity or presence may be assayed using the processes and probes of the present invention include small molecules, and enediynes.

In certain embodiments, the nucleic acid cleavage agent cleaves the probe in the single stranded portion of the stem loop structure. In other embodiments, the nucleic acid cleavage agent cleaves the probe in the double stranded portion of the stem loop structure. In yet other embodiments, the nucleic acid cleavage agent cleaves the probe in at the junction of the single stranded portion and the double stranded portions of the stem loop structure.

In certain embodiments, the fluorophore and quencher are internally coupled to the probe. In certain other embodiments, the fluorophore and quencher are coupled to the 5′ and/or 3′ ends of the probe.

In certain embodiments, the nucleic acid cleavage agent cleaves the probe at a site between the quencher and the fluorophore.

In some embodiments, probes of the present invention are immobilized to a solid surface.

In certain embodiments, the probe comprises a recognition site specific for a nucleic acid cleavage agent. In certain embodiments, the recognition site is located in the single stranded portion of the stem loop structure. In other embodiments, the recognition site is located in the double stranded portion of the stem loop structure. In yet other embodiments, the recognition site spans the junction between the single stranded and the double stranded portions of the stem loop structure. In certain embodiments, the recognition site is located at a site between the quencher and the fluorophore.

The present invention also provides processes for evaluating activity of a nucleic acid cleavage agent. In certain embodiments, the processes comprise, the process comprising a. incubating the nucleic acid cleavage agent with a first probe, the first probe comprising an oligonucleotide that forms a stem loop structure and having a first sequence, a fluorophore, and a quencher, wherein the fluorophore and the quencher are positioned such that the fluorophore fluoresces less when the probe is intact than when the probe is cleaved; b. measuring level of the fluorescence of the first probe; c. incubating the nucleic acid cleavage agent with a second probe, the second probe comprising an oligonucleotide that forms a stem loop structure and having a second sequence, a fluorophore, and a quencher, wherein the fluorophore and the quencher are positioned such that the fluorophore does not fluoresce when the probe is intact and does fluoresce when the probe is cleaved; d. measuring level of the fluorescence of the second probe; comparing the level of fluorescence of the first probe to the level of fluorescence of the second probe; and correlating the amount of fluorescence of the first and second probes with activity of the nucleic acid cleavage agent.

In certain embodiments, cleavage of each probe is carried out in a separate reaction vessel. In other embodiments, cleavage of more than one probe is carried out in the same reaction vessel, and, preferably, each type of probe is linked to a different fluorophore, and the fluorophores are distinguishable from one another.

The present invention also provides processes for evaluating activity of a nucleic acid cleavage agent. In certain embodiments, the processes comprise: a. incubating the nucleic acid cleavage agent with a probe in a first set of conditions, the probe comprising an oligonucleotide that forms a stem loop structure, a fluorophore, and a quencher, wherein the fluorophore and the quencher are positioned such that the fluorophore fluoresces less when the probe is intact than when the probe is cleaved; b. measuring level of fluorescence of the probe in the first set of conditions; c. incubating the nucleic acid cleavage agent with the probe in a second set of conditions; d. measuring level of fluorescence of the probe in the second set of conditions; comparing the level of fluorescence of the probe in the first set of conditions to the level of fluorescence of the probe in the second set of conditions; and correlating the level of fluorescence in the first and second conditions to the activity of the nucleic acid cleavage agent.

The present invention also provides processes for evaluating the effectiveness of a nucleic acid protective agent (also called a “nucleotide protective agent”). In certain embodiments, the process comprises: a. incubating a nucleic acid cleavage agent and a probe, the probe comprising an oligonucleotide that forms a stem loop structure, a fluorophore, and a quencher, wherein the fluorophore and the quencher are positioned such that the fluorophore fluoresces less when the probe is intact than when the probe is cleaved; b. measuring the level of fluorescence of the probe as incubated in step (a); c. incubating the nucleotide protective agent, the nucleic acid cleavage agent, and the probe; d. measuring the level of fluorescence of the probe as incubated in step (c); e. comparing the levels of fluorescence measured in steps (b) and (d); and f. correlating amount of difference in the fluorescence levels measured in steps (b) and (d) with the effectiveness of the nucleic acid protective agent.

Examples of protective agents that may be studied using processes and probes according to the present invention include histones and transcription factors, as well as other proteins, peptides, small molecules, and other molecules that interact with nucleic acids.

The present invention provides oligonucleotide probe useful for assaying the activity, presence, efficiency, and the like of nucleic acid cleavage agents and protective agents. In certain embodiments, probes according to the present invention comprise a. an oligonucleotide that forms a stem loop structure and comprises a recognition site for a nucleic acid cleavage agent; b. a fluorophore, and c. a quencher, wherein the fluorophore and the quencher are positioned such that the fluorophore does not fluoresce when the probe is intact and does fluoresce when the probe is cleaved.

Kits comprising at least one probe according to the innention are also provided. Kits may also comprise at least one nucleic acid cleavage agent that recognizes the recognition site.

In certain embodiments, the cleavage agent and the recognition site are known to bind or otherwise interact. In certain preferred embodiments, the invention provides methods and reagents (such as oligonucleotides) for assessing the titer of cleavage agents in, for example, a solution, sample, or organism. In a particularly preferred embodiment, the invention provides methods and reagents for assessing the titer of cleavage agents, such as calicheamicin, in fermentations of bacteria, such as Micromonospora.

In other embodiments, it is unknown whether or how strongly the recognition sequence and the cleavage agent bind or otherwise interact.

In preferred embodiments, interaction of the cleavage agent and the recognition site results in scission of the oligonucleotide. In preferred embodiments, this scission leads to immediate separation of the fluorophore-quencher pair and results in a spontaneous fluorescent signal which directly correlates to the extent of nucleotide cleavage.

### BRIEF DESCRIPTION OF THE DRAWINGS

FIG. 1. Non-enzymatic DNA-cleaving agents: calicheamicin γ₁^{I} from M. echinospora (A), esperamicin A₁ from A. verrucosospora (B), bleomycin from S. verticillus (C), methidiumpropyl-Fe⁺²-EDTA (MPE, D) and Fe⁺²-EDTA (E).

FIG. 2. A schematic diagram of molecular beacons, molecular break lights and the specific break lights used in this study. The solid lines represent covalent bonds, dashed lines represent hydrogen bonding, letters represent arbitrary bases, the gray shaded ball represents the fluorophore (FAM), the black ball represents the corresponding quencher (DABCYL) and the dashed wedges represent fluorescence. (B) Principle of operation of molecular break lights. Cleavage of the stem by an enzymatic or non-enzymatic nuclease activity results in the separation of the fluorophore-quencher pair and a corresponding fluorescent signal. (C) Molecular break lights used in Examples. The stem of break light A contains a preferred calicheamicin recognition site (bold-faced) and the stem of break light B carries the BamHI recognition site (bold-faced). The predicted cleavage sites are illustrated by arrows.

FIG. 3. The observed change in fluorescence intensity over time of an assay containing 3.2 nM break light at 37° C. (a) Break light A with 100 U BamHI (□), break light B with 100 U BamHI (∘) and break light B without enzyme (●) (10 mM TrisHCl, 50 mM NaCl, 10 mM MgCl₂, 1 mM DTT, pH 7.9; λ_{Ex}=485 nm, λ_{Em}=517 nM). (b) Break light A with and 10 U DNaseI (□), break light B with 10 U DNaseI (∘) and break light A without enzyme (●) (40 mM Tris HCl, 10 mM MgSO₄, 1 mM CaCl₂, pH 8.0; λ_{Ex}=485 nm, λ_{Em}=517 nM).

FIG. 4. The determination of BamHI steady state kinetic parameters using break light B. (a) The observed change in fluorescence intensity over time of an assay containing a constant 3.2 nM break light B at 37° C. (6 mM TrisHCl, 100 mM NaCl, 6 mM MgCl₂, 1 mM DTT, pH 7.5; λ_{Ex}=485 nm, λ_{Em}=517 nM), BamHI (10 U) and varying non-labeled substrate oligonucleotide. Total substrate concentrations (including break light): 389 nM (∘), 196 nM (□), 81 nM (⋄), 42 nM (Δ), 11 nM (●), 7.5 nM (▪) and 3.4 nM (♦). (b) Lineweaver-Burke plot from FIG. (4a) after correction for the carrier dilution effect.

FIG. 5. Cleavage of break light A by calicheamicin and esperamicin. The observed DNA cleavage over time of an assay containing 3.2 nM break light A at 37° C. (40 mM Tris.HCl, pH 7.5, λ_{Ex}=485 nm, λ_{Em}=517 nM), DTT (50 μM) and varied enediyne. (a) Calicheamicin concentrations: 31.7 nM (∘), 15.9 nM (□), 3.2 nM (⋄), 1.6 nM (Δ), 0.78 nM (●) and 0.31 nM (▪). (b) Esperamicin concentrations: 31.7 nM (∘), 15.9 nM (□), 3.2 nM (⋄), 1.6 nM (Δ), 0.78 nM (●), 0.31 nM (▪) and 0.15 nM (♦).

FIG. 6. Cleavage of break light A by Fe⁺²-dependent agents. (a) The observed DNA cleavage over time of an assay containing a constant 3.2 nM break light A at 37° C. (50 mM sodium phosphate, 2.5 mM ascorbate, pH 7.5; λ_{Ex}=485 nm, λ_{Em}=517 nM) and varied bleomycin. Bleomycin concentrations: 200 nM (∘), 100 nM (□), 50 nM (⋄) 25 nM (Δ), 12.5 nM (●), 5 nM (▪) and 2.5 nM (▴). (b) The observed DNA cleavage over time of an assay containing a constant 3.2 nM break light A at 37° C. (40 mM Tris HCl, 2.5 mM ascorbate, pH 7.5; λ_{Ex}=485 nm, λ_{Em}=517 nM) and varied MPE. Fe(II) concentrations: 8 μM (∘), 4 μM (□), 2 μM (⋄), 1 μM (Δ), 500 nM (●), 250 nM (▪) and 125 nM (▴). (c) The observed DNA cleavage over time of an assay containing a constant 32 nM break light A at 37° C. (40 mM Tris HCl, 2.5 mM ascorbate, pH 7.5; λ_{Ex}=485 nm, λ_{Em}=517 nM) and varied MPE. Fe(II) concentrations: 50 nM (∘), 125 nM (□), 250 nM (⋄), 500 nM (Δ), 1 μM (●) and 2 μM (▪). (d) The observed DNA cleavage over time of an assay containing a constant 32 nM break light A at 37° C. (40 mM TrisHCl, 2.5 mM ascorbate, pH 7.5; λ_{Ex}=485 nm, λ_{Em}=517 nM) and varied Fe⁺²-EDT A. Fe(II) concentrations: 12.5 μM (∘), 6.3 μM (□), 3.1 μM (⋄), and 1.3 μM (Δ).

FIG. 7A is a graph of the UV-visible absorption spectra of purified mbp-CalC. The purified mpb-CalC was analyzed in the following solution: 52 μM mpb-CalC; 10 mM Tris-HCl, pH 7.5). The inset shows the results of low temperature (4.3 K) the X-band EPR analysis of CalC. 250 μM mpb-CalC containing 0.5 mol Fe per mol CalC was analyzed in 10 mM Tris-HCl, pH 7.5. The spectrometer settings were as follows: field set=2050 G; scan range=4,000G; time constant=82 s; modulation amplitude=16 G; microwave power=31 μW; frequency=9.71 Ghz; gain=1000; determined spin quantitation=90±10 μM Fe.

FIG. 7(b) is a photograph of an ethidium bromide stained agarose gel. Lane A: calicheamicin, no DTT; lane B: DTT, no calichearnicin; lane C: DTT and calicheamicin; lane D: DTT, calicheamicin, and mbp; lane E: calicheamicin, DTT, and apo-mbp-CalC (which lacks the Fe cofactor); lane F: DTT, calicheamicin, and mbp-CalC; and lane G: calicheamicin, DTT, and apo-mbp-CalC, preincubation with 1 mM FeSO₄ (Fe⁺²) or FeCl₃ (Fe⁺³) prior to the activity assay.

FIG. 8 is a schematic diagram of the first continuous assay for enediyne-induced DNA cleavage, the Molecular Break Lights. The solid lines represent covalent bonds, dashed lines represent hydrogen bonding, letters represent arbitrary bases, the gray shaded ball represents the fluorophore (FAM: fluorescein), the black ball represents the corresponding quencher (DABCYL:4-(4-′demethylaminophenylazo)-benzoic acid) and the dashed wedges represent fluorescence.

FIG. 9 shows the direct in vitro inhibition of calicheamicin-mediated DNA cleavage using the break light assay. 3.6 pM break light A is coincubated with 3.5 nM calicheamicin with increasing amounts of CalC. Complete inhibition of calicheamicin is achieved with roughly 2-fold excess of CalC. CalC has no effect on esperamicin-induced cleavage of DNA.

### DETAILED DESCRIPTION OF THE INVENTION

Previous assays for enediyne cleavage of nucleotides relied upon discontinuous assays using radioactive nucleotide probes, electrophoresis and subsequent phosphoimager analysis. In contrast, by using methods and reagents (molecular break lights) of the present invention, one can directly follow the extent of nucleotide cleavage by a specific enediyne in real time with high sensitivity.

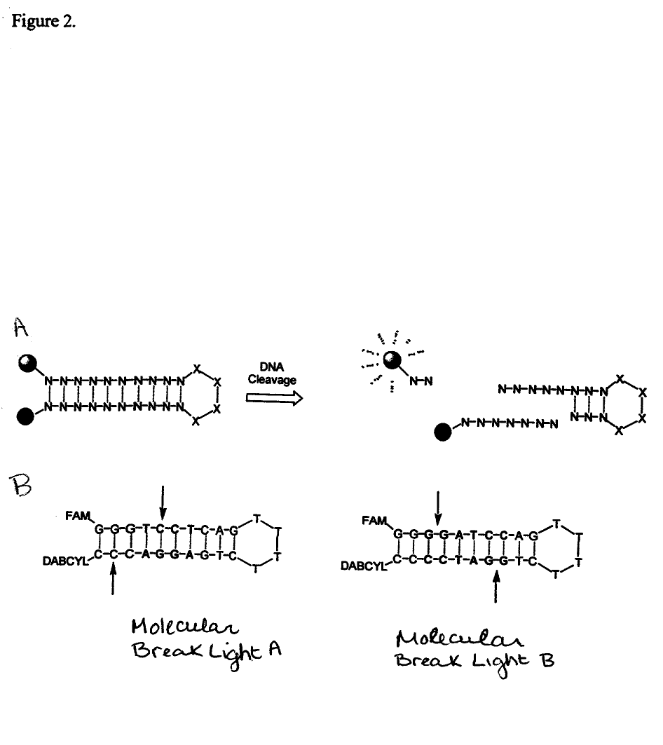

The present invention provides a modified hairpin-forming oligonucleotide to continuously assess nucleotide cleavage by enediynes and other nucleic acid cleavage agents. These oligonucleotides are also useful for continuous assessment of protection of nucleotides from cleavage agents. An exemplary substrate oligonucleotide probe (or molecular break light) for assaying oligonucleotide cleavage is a single-stranded oligonucleotide which adopts a stem-and-loop structure and carries a 5′-fluorescent moiety and a 3′-non-fluorescent quenching moiety. (FIG. 2A). The stem design keeps these two moieties in close proximity to each other to provide fluorescence quenching by fluorescence resonance energy transfer (FRET) and also includes a nucleotide-binding recognition sequence for a nucleic acid cleavage agent of interest. (FIG. 2A). Thus, the quenching is intramolecular.

Scission of the stem of the probe by a nucleic acid cleavage agent leads to ” separation of the two moieties of the fluorophore-quencher pair. Separation of the moieties results in a spontaneous fluorescent signal which directly correlates to the extent of nucleotide cleavage. Preferably, the separation and fluorescence occur substantially simultaneously with the scission. The hairpin-forming oligonucleotide probes of the present invention may be referred to as “molecular break lights” (as in nucleotide strand “break”).

As the fluorescent signal is preferably visible immediately upon cleavage of a molecular break light probe, cleavage events can be observed in real time. Therefore, molecular break light probes according to the present invention are useful for continuous monitoring of continuous enzymatic and small molecule-catalyzed nucleotide cleavage events.

As molecular break lights comprise both single- and double-stranded DNA or RNA, cleavage sites can be located in either type of nucleotide. Single strand cleavage sites may be located in the loop, and double strand cleavage sites may be located in the stem. Therefore, the molecular break lights of the present invention provide for the assessment of cleavage by both agents that cleave single-stranded nucleotides and agents that cleave double-stranded nucleotides.

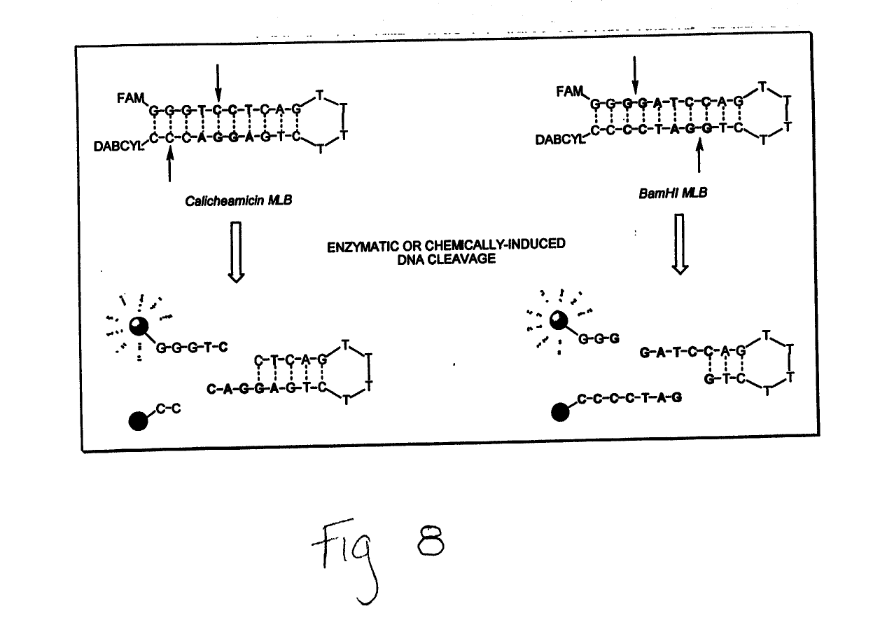

Generally, molecular beacons operate by a separation of the fluorophore-quencher pair resulting in a corresponding fluorescent signal. Molecular break lights, as illustrated in the FIG. 8, operate through cleavage of the stem by an enzymatic or non-enzymatic nuclease activity resulting in the separation of the fluorophore-quencher pair and corresponding fluorescent signal. In FIG. 8, the molecular break lights contain either a preferred calicheamicin recognition site (bold-faced, TCCT) or the BamHI recognition site (bold-faced, GGATCC). The predicted cleavage sites are illustrated by arrows.

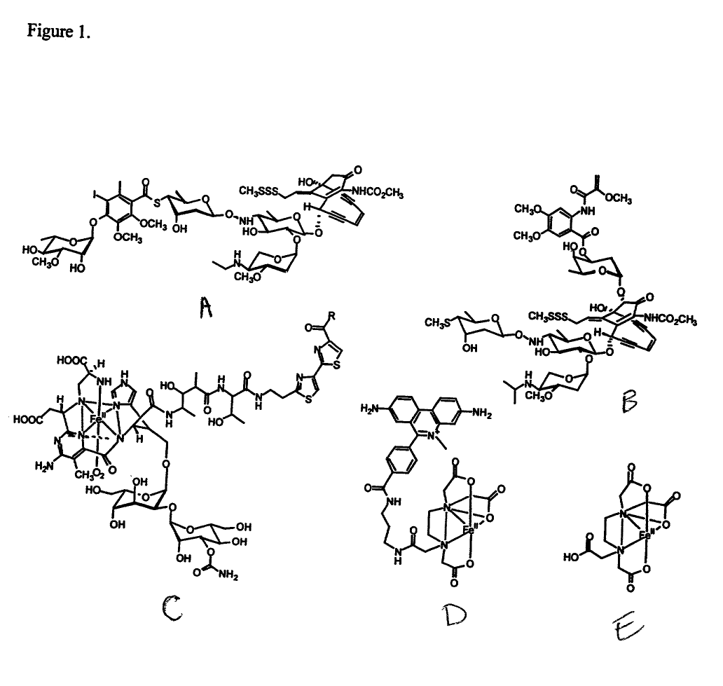

The break light assay has broad, general utility. The break light assay is useful for the analysis of nucleotide cleavage by, as non-limiting examples, random nucleases, sequence specific nucleases, context specific nucleases, and small molecules. For example, the break light assay can provide a direct comparison of the cleavage efficiencies by different agents. A comparison of the cleavage efficiencies of naturally-occurring enediynes in FIG. 1 (calicheamicin, A, and esperamicin, B), non-enediyne small molecule agents (bleomycin, C, methidiumpropyl-Fe-EDTA, D, and Fe-EDTA, E) as well as the restriction endonuclease BamHI is discussed further herein.

The molecular break light assay is advantageous over previous FRET-based DNA cleavage assays in that one can achieve a significantly higher signal to noise ratio (˜40) with molecular break lights, in comparison to assays based upon oligonucleotide pairs with a single oligonucleotide substrate, which have a much lower signal to noise ratio (˜2). See, e.g., Tyagi, S., et al. Nature BiotechnoL 14: 303-308 (1996); Tyagi, S., et al. Nature Biotechnol. 16: 49-53 (1998).

Furthermore, the molecular break light assay exceeds the sensitivity of assays based upon fluorescence correlation spectroscopy (FCS), is a very sensitive technique, by greater than 10-fold. Additionally, FCS requires extremely specialized instrumentation. See, e.g., Kettling, U., et al. Proc. Natl. Acad. Sci. USA 95: 1416-1420 (1998). Such specialized instrumentation is not required to perform the assays of the present invention.

The sensitivity of assays according to the present invention also rival the typical discontinuous assay for detection of DNA-damaging agents known as the biochemical induction assay (BIA). Given the simplicity, speed and sensitivity of the present inventive approach, the inventive methodology can be extended to a high throughput format and become a new method of choice in modem drug discovery to screen for novel protein-based or small molecule-derived DNA cleavage agents.

Nucleotide-protecting agents (e.g., transcription factors, histones, etc.) prevent or reduce cleavage by cleaving agents. Unlike prior assays, the molecular break lights of the present invention may also be used to assess the protection by various nucleotide-protecting agents of oligo- or polynucleotides from cleavage. For example, as discussed further herein, the protection from cleavage by calicheamicin that is conferred by the protein CalC can be measured using assays and reagents according to the present invention. The protective action of any nucleotide-protecting agent (protein or other) may likewise be measured. Whether, or to what degree, a nucleotide-protecting agent of interest protects an oligo- or polynucleotide from cleavage by a nucleic acid cleavage agent of interest may be observed and measured by comparing (a) the cleavage of molecular break light probes by the nucleotide cleavage agent of interest in the presence of the nucleotide-protecting agent of interest with (b) the cleavage of molecular break lights by the nucleic acid cleavage agent of interest without the addition of the nucleotide-protecting agent of interest. The amounts of nucleic acid cleavage agent of interest and nucleotide-protecting agent of interest may be varied. Molecular break lights may also be the most sensitive and the first continuous assay for such systems.

Molecular break light probes having nucleotide binding sequences specific for nucleic acid cleavage agents of interest may be made using art-known techniques, e.g., for manipulating nucleotides. As molecular break lights comprise both single- and double-stranded DNA or RNA, cleavage sites can be located in either type of nucleotide. Single strand cleavage sites may be located in the loop, and double strand cleavage sites may be located in the stem. Therefore, the molecular break lights of the present invention provide for the assessment of cleavage by both agents that cleave single-stranded nucleotides and agents that cleave double-stranded nucleotides.

The oligonucleotide sequences of molecular break lights probes according to the present invention may be DNA, RNA, peptide nucleic acid (PNA) or combinations thereof. Modified nucleotides may be included, for example nitropyrole-based nucleotides or 2′-O-methylribonucleotides. Modified linkages also may be included, for example phosphorothioates. Thus, molecular break lights probes may be designed and used to assay cleavage by nucleic acid cleavage agents specific for nucleotide sites containing a wide array of nucleotides.

A wide range of fluorophores may be used in probes and primers according to this invention. Available fluorophores include coumarin, fluorescein, tetrachlorofluorescein, hexachlorofluorescein, Lucifer yellow, rhodamine, BODIPY, tetramethylrhodamine, Cy3, Cy5, Cy7, eosine, Texas red and ROX. Combination fluorophores such as fluorescein-rhodamine dimers, described, for example, by Lee et al. (1997), Nucleic Acids Research 25:2816, are also suitable. Fluorophores may be chosen to absorb and emit in the visible spectrum or outside the visible spectrum, such as in the ultraviolet or infrared ranges.

Preferable fluorophores for use in the present invention include any fluorophore that has strong absorption in the wavelength range of the available monochromatic light source. For example, when an argon laser emitting blue light (488 nm) or a blue light emitting diode is used as the excitation source, fluorescein can serve as an excellent fluorophore. Another fluorophore that is efficient in the blue range is 3-(ε-carboxy-pentyl)-3′-ethyl-5,5′-dimethyloxacarbocyanine (CYA). For these harvester fluorophores, the emitter fluorophores can be 2′,7′-dimethoxy-4′,5′-dichloro-6-carboxy-fluorescein (JOE), tetrachlorofluorescein (TET), N,N,N′,N′-tetramethyl-6-carboxyrhodamine (TAMRA), 6-carboxy-X-rhodamine (ROX), Texas red, and a number of cyanine dyes whose absorption spectra share substantial spectral overlap with the emission spectrum of fluorescein and CYA. With sources of different wavelengths, fluorphores may be selected to absorb and emit anywhere along the spectrum from ultraviolet to infrared. Compound fluorophores can also be used as a fluorophore.

A quencher is a moiety that, when placed very close to an excited fluorophore, causes there to be little or no fluorescence. Suitable quenchers described in the art include particularly DABCYL and variants thereof, such as DABSYL, DABMI and Methyl Red. Fluorophores can also be used as quenchers, because they tend to quench fluorescence when touching certain other fluorophores. Exemplary quenchers include chromophores such as DABCYL or malachite green, and fluorophores that do not fluoresce in the detection range when the probe is intact.

Preferred embodiments of these probes, labeled with a fluorophore and a quencher, are “dark” (that is, have relatively little or no fluorescence) when intact, but fluoresce when cleaved. Preferably, the total fluorescence of preferred probes when intact is less than twenty percent of their total fluorescence when cleaved. Most preferably, the quencing is complete≠the fluorophore does not fluoresce when the probe is intact.

When the probe is intact, the moieties of the FRET pair are in a close, quenching relationship. Most preferably the two moieties touch each other. However, separation by a single base pair along the stem duplex is almost always satisfactory. Even greater separations are possible in many instances, namely, 2-4 base pairs or even 5-6 base pairs. For these greater separations the helical nature of the stem duplex should be considered for its effect on the distance between the moieties.

Fluorophores and quenchers can be added to the probe by functionalization of the appropriate building blocks (e.g., deoxyribonucleotides) such that the fluorophores will be present on the building blocks prior to the formation of the probe, or they may be conjugated to the probe after formation, as appropriate. Various chemistries known to those of average skill in the art can be used to ensure that the appropriate spacing between the fluorophore and the quencher is obtained. In addition fluorophore phosphoramidites, for example a fluorescein phoshoramidite, can be used in place of a nucleoside phosphoramidite. A nucleotide sequence that contains such a substitution is considered to be an “oligonucleotide” as that term is used in this disclosure and in the appended claims, despite the substitution.

Fluorophores and quenchers can be attached via alkyl spacers to different positions on a nucleotide. The labels can be placed at internal or terminal locations in the oligonucleotide, using commonly available DNA synthesis reagents. The labels can also be placed at internal positions in oligonucleotides by substituting a nucleotide linked to a fluorophore moiety during synthesis. Although, commonly available spacers that employ alkyl chains of several carbons (Glen Research) can be used successfully, the degree of quenching and the extent of energy transfer can be further optimized by varying the length of the spacers.

Molecular break light probes are useful in many situations. For example, where the nucleic acid recognition or binding sequence for which a cleavage agent of interest is specific is known, molecular break light probes having that sequence may be produced and used, e.g., to analyze the cleavage rate of the cleavage agent alone or in the presence of nucleic acid protection agents. As another example where the nucleic acid recognition or binding sequence for which a cleavage agent of interest is specific is known, molecular break light probes having that sequence may be produced and used, e.g., to assess the titer of cleavage agents in, for example, a solution, sample, or organism.

As a specific example, the presence or titer of cleavage agents, such as calicheamicin, in fermentations of bacteria, such as Micromonospora, may be assessed using molecular break light probes according to the present invention. Where it was desired to know whether calicheamicin was present in a sample, a molecular break light probe having a recognition sequence for calicheamicin could be incubated with the sample. An increase of fluoresence over background would indicate the presence of calicheamicin. As the rate of molecular break light cleavage is dependnet upon the concentration of the cleavage agent, the concentration of calecheamicin in the sampole can be assessed by comparing the observed rate to rates of known concentrations of calicheamicin, e.g., standard curves.

As another example, where it is known that a specific the recognition sequence and a cleavage agent of interest bind or otherwise interact, but it is not known how strongly they interact or the rate and/or efficiency at which cleavage occurs, molecular break light probes having the recognition sequence may be produced and used to asses the interaction strength, efficiency, and/or speed.

Probes and processes according to the present invention also find use, e.g., when it is desirable to determine the optimal conditions for the activity of a nucleic acid cleavage agent. In such embodiments, the cleavage of a single type of molecular break light probe by a nucleic acid cleavage agent of interest is evaluated under different conditions. Conditions that can be varied include temperature, pH, buffer and salt concentraions, cofactor concentrations, and the like. Other parameters of interest will be readily apparent to the skilled artisan.

Likewise, probes and processes according to the present invention also find use, e.g., when it is desirable to determine the recognition site for a nucleic acid cleavage agent, to assess the specificity of a nucleic acid cleavage agent for a given recognition site, or to compare the efficiencies of cleavage by a nucleic acid cleavage agent at different recognition sites. Cleavage of recognition sites having the same sequence but differing location on the probe may also be assessed. In such embodiments, a probe comprising each recognition site or potential recognition site of interest is prepared. Cleavage efficiencies and rates of the probes by a cleavage agent of interest are determined and compared.

Where it is desired to evaluate simultaneously the cleavage of more than one type of probe by a common nucleic acid cleavage agent, the probes may be incubated with the cleavage agent in separate vessels. However, as many different, distinguishable fluorophores are known in the art, it may be desirable to couple each type of probe to a different fluorophore. The use of distinguishable fluorophores enables the researcher to evaluate simultaneously the cleavage of more than one type of probe by a common nucleic acid cleavage agent in a single reaction vessel.

In certain embodiments, cleavage of each probe is carried out in a separate reaction vessel. In other embodiments, cleavage of more than one probe is carried out in the same reaction vessel, and, preferably, each type of probe is linked to a different fluorophore, and the fluorophores are distinguishable from one another.

As yet another example, where the recognition site for a cleavage agent of interest is unknown, the cleavage agent can be tested on many different molecular break light probes, each having a different recognition sequence. Similarly, where it is desired to determine a cleavage agent that will cleave a recognition sequence of interest, various cleavage agents may be tested for cleavage of a molecular break light probe having the recognition sequence of interest.

In assays wherein cleavage of multiple different molecular break light probes is assessed, several different probes may be assayed in a may be used in a single reaction tube or other container for multiplex assays by coupling each different molecular break light probe to a different fluorophore, each of which can be distinguished from the others under the assay conditions.

Molecular break light probes according to the present invention may also be coupled to substrates. For example, microarray technology may be used to immobilize different types of probes to discrete, known locations on a substrate. The positional data generated by the microarray facilitates the assessment of the cleavage of more than one type of probe by a cleavage agent. Where probes of different types are immobilized at known locations, the use of different fluorophores to distinguish types of probes is not necessary, but may be used to further increase the number of types of probes that may me simultaneously studied.

Molecular break lights probes find particular use in the comparison of enzymatic and non-enzymatic nucleic acid cleavage agents. Non-enzymatic cleavage agents such as calicheamicin are essentially involved in single turnover events and, thus, their direct comparison to an enzyme-catalyzed event is difficult. In fact, significant controversy exists regarding the more simplistic comparison of synthetic and biological catalysts in general. See, e.g., Jacobsen, E. N. et al. Chem. Biol. 1: 85-90 (1994).

The cleavage efficiencies of naturally-occurring enediynes in FIG. 1 (calicheamicin, A, and esperamicin, B), non-enediyne small molecule agents (bleomycin, C, methidiumpropyl-Fe-EDTA, D, and Fe-EDTA, E) as well as the restriction endonuclease BamHI were assessed and compared.

Enzymatic Cleavage as Proof of Principle.

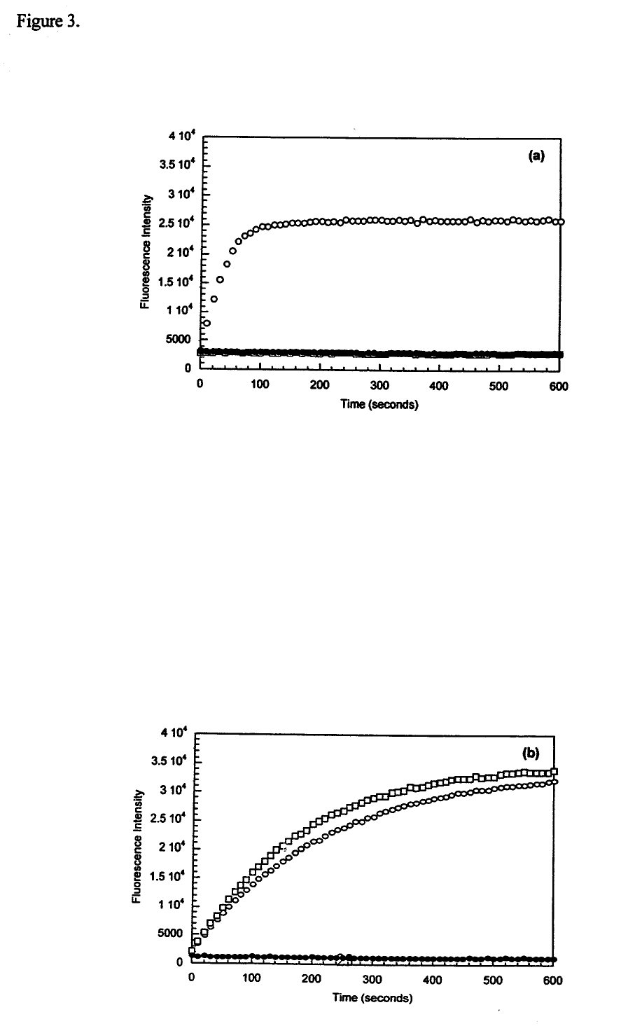

The specificity of the designed molecular break lights via enzymatic cleavage was demonstrated in an assay using BamHI. Only break light B (FIG. 2B, specific for BamHI) should cleave in the presence of the restriction endonuclease BamHI while both break light A (FIG. 2B, specific for calicheamicin) and break light B should be digested by the non-specific nuclease DNaseI. As anticipated, FIG. 3a reveals a time dependent and [BamHI]-dependent increase of fluorescence only with B while A shows no change at 37° C. (FIG. 3b) illustrates an increase of fluorescence over time with either break light A or B when digested with DNaseI which is also [DNaseI]-dependent. In comparison, control samples containing break lights alone or break lights in the presence of BSA gave no change in fluorescence over >2 hr at 37° C. Given the lack of fluorescence in the absence of enzyme, the designed break lights show no appreciable melting at the designated assay temperature. Furthermore, these experiments clearly demonstrate the specificity of cleavage by BamHI for break light B and illustrate the principle application of molecular break lights to assess DNA cleavage.

Interestingly, the fluorescence maximum intensity obtained upon complete BamHI cleavage was only 75% that observed in the presence of DNaseI at the same concentration of molecular break light. Furthermore, after the BamHI reaction was complete, the addition of BamHI showed no change while the addition of DNaseI resulted in additional cleavage to give the expected 100% fluorescence maximum. This observation suggests the poly-guanidine tail left attached to FAM upon BamHI digestion quenches the fluorescent signal by ˜25%. Consistent with this finding, PAGE analysis of the reaction products confirmed the presence of a 3-base overhang after excess treatment with BamHI which is completely degraded upon DNaseI digestion. As a result, the fluorescence maximum observed with excess BamHI was designated 100% cleavage for the BamHI kinetic studies described below.

BamHI steady state kinetic determination and sensitivity limits were also assessed. While continuous assays for non-specific nucleases have been based upon ΔA₂₆₀ as a function of cleavage of generic chromosomal DNA (e.g. sonciated herring sperm DNA), only a few examples of continuous restriction endonuclease assays have been reported. Thus, most restriction endonuclease steady-state kinetic determinations have relied upon discontinuous assays using radioactive DNA probes, electrophoresis and subsequent phosphoimager analysis. To demonstrate the utility of molecular break lights for this application, the steady-state kinetic parameters for a commercially available BamHI were determined. In the present assay, the dependence of BamHI hydrolysis on substrate concentration was investigated using mixtures of a fixed amount of B and varying amounts of an analogous non-labeled oligonucleotide (lacking both FAM and DABCYL) over a wide substrate concentration range. The apparent competitive inhibition observed due to phenomenon of “carrier dilution” was corrected to give the appropriate kinetic parameters as previously described. See, e.g., Roy, K. B., et al. Anal. Biochem. 220: 160-164 (1994).

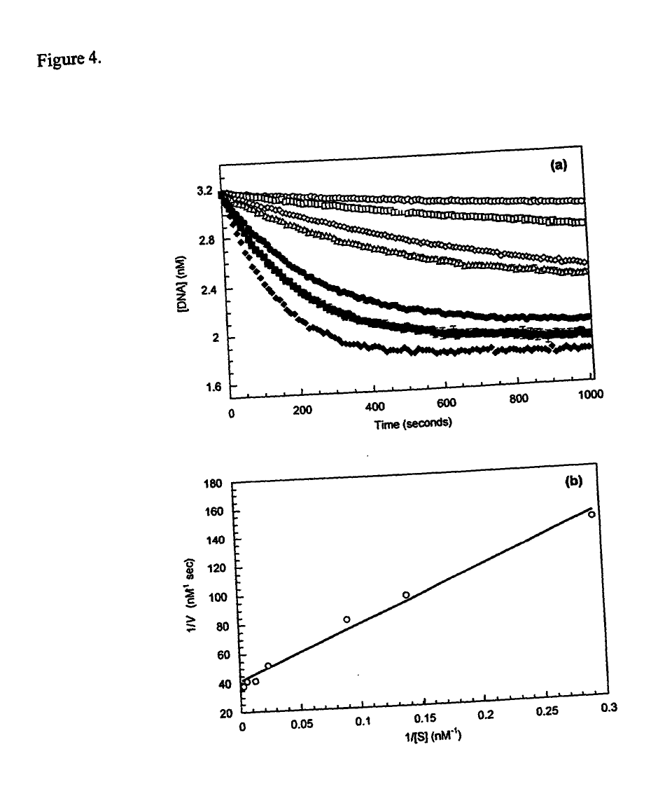

As illustrated in FIG. 4a, the velocity curves decrease with an increase in initial substrate concentration although the true velocity has actually increased, due to the carrier dilution by the non-labeled oligonucleotide. The observed velocity (Vₐₚₚ) is related to the actual velocity (V_{act} by equation [I] where [S_{act}] and [S*] are the total substrate concentration and B concentration, respectively. The reciprocal plot after correction for this phenomenon is illustrated in FIG. 4b.

From FIG. 4b, the determined Kₘ=8.9±0.5 nM and v max=0.024±0.001 nM sec⁻¹. While these values differ slightly from previously reported values for BamHI of Kₘ=0.4 nM and V max=0.009 nM sec⁻¹, kinetic parameters of restriction endonucleases vary significantly depending upon the oligonucleotide substrate. It should be acknowledged that our examination of three different commercial sources of BamHI (Promega, New England Biolabs and GIBCO BRL) gave markedly distinct specific activities (ranging roughly an order of magnitude). Thus, the differences in the reported kinetic parameters could also simply reflect distinctions in the enzyme preparation and/or commercial assay buffers. Most importantly, the utility of molecular break lights to assess the kinetic parameters of enzymatic DNA cleavage has been demonstrated. Furthermore, it is expected this approach could be directed toward any endonuclease by simply changing the recognition sequence found within the molecular break light stem.

A recent fluorescence correlation spectroscopy (FCS) assay for the restriction endonuclease EcoRI using 0.8 nM of dual fluorophoric-labeled dsDNA and a highly specialized FCS spectrometer, reported a detection limit of 1.6 pM EcoRI. Kettling, U., et al. Proc. Natl. Acad. Sci. USA 95: 1416-1420 (1998). Under the conditions containing even slightly less oligonucleotide (0.68 nM molecular break light), cleavage was easily detectable to 3.7 pM BamHI. Furthermore, due to the significantly low signal to noise of this assay, increasing the molecular break light concentration (34 nM) lowered the detection limit into the fM range (0.12 pM BamHI).

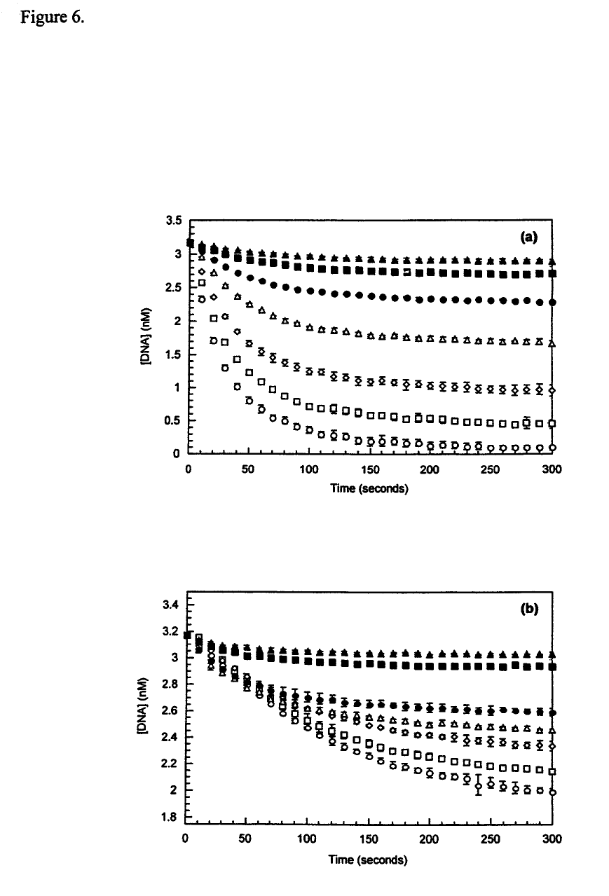

Enediyne-catalyzed cleavage was also assessed. Previous assays for enediyne cleavage of DNA relied upon discontinuous assays using radioactive DNA probes, electrophoresis and subsequent phosphoimager analysis. In contrast, by using the molecular break lights of the present invention, one can directly follow the extent of DNA cleavage by a specific enediyne in real time with high sensitivity. To demonstrate, FIG. 5a and FIG. 5b illustrate enediyne concentration dependent cleavage of break light A with either calicheamicin or esperamicin in the presence of excess reductive activator DTT. Under the conditions described, this assay allows the detection of calicheamicin in the pM range. This sensitivity compares to that of the biochemical induction assay (BIA), the method of choice in detecting DNA-damaging agents. See, e.g., Roy, K. B., et al. Anal. Biochem. 220: 160-164 (1994). Furthermore, the sensitivity can be significantly enhanced by simply increasing the concentration of the molecular break light in the assay as demonstrated with the iron-dependent agents. The observed maximum fluorescence obtained upon cleavage of 3.2 nM break light A with either calicheamicin or esperamicin was identical to that observed with DNaseI, consistent with complete degradation of the oligonucleotide. As controls, incubation of molecular break light A with either DTT or enediyne alone revealed no change in fluorescence. Furthermore, although there is some debate regarding the “specificity” of calicheamicin, break light B was cleaved by calicheamicin at an identical rate. This supports the view that the specificity of calicheamicin is more dependent upon context and perhaps less so on DNA sequence. It should also be noted that calicheamicin leads to predominately double-stranded cleavage while esperamicin provides single-stranded nicks and the current molecular break light assay can not distinguish these two phenomena.

Interestingly, two distinct rates were observed in the enediyne molecular break light assay. The first (0-50 seconds) is a lag time most likely attributed to the enediyne activation while the second (50-200 seconds) is indicative to the initial velocity of DNA cleavage. To confirm this, assays were also established in which DTT and enediyne were first preincubated for 1-5 min followed by initiation via the addition of the substrate oligonucleotide. In these preincubation experiments, the previously observed “lag time” attributed to activation was no longer evident while the initial velocity of DNA cleavage was identical to that determined in the standard assay. Preincubation for longer periods (>30 min) revealed the same phenomenon, suggesting “activated” enediynes are perhaps more stable in an aqueous aerobic environment than previously estimated. See, e.g., Thorson, J. S., et al. Bioorgan. Chem. 27: 172-188 (1999).

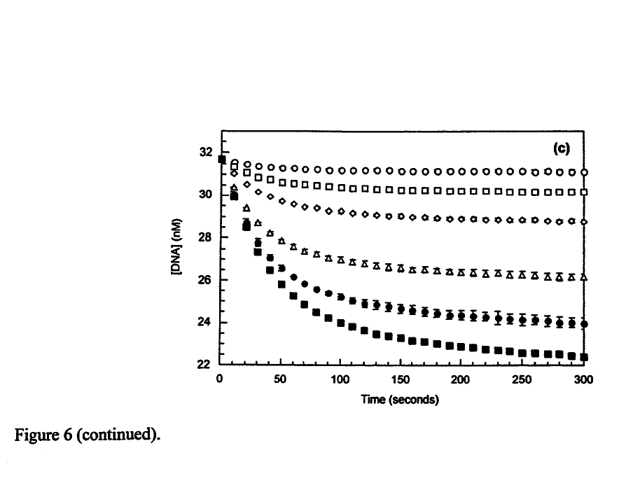

Cleavage catalyzed by Fe⁺²-dependent agents was assessed. To further demonstrate the utility of molecular break lights, the ability to assess DNA cleavage catalyzed by Fe⁺²-dependent agents was investigated. The agents selected include the natural metabolite from Streptomyces verticillus, bleomycin, FIG Ic, and two DNA-footprinting reagents, methidiumpropyl-Fe-EDTA (MPE), FIG. 1d, and Fe-EDTA, FIG. 1e. While the precise mechanism of DNA cleavage by bleomycin is still controversial, MPE and Fe⁺²-EDTA cleave DNA via the generation of diffusable hydroxy radicals which ultimately contribute to oxidative DNA cleavage. Of these three, bleomycin also contains a strong minor groove binding constituent while MPE carries a DNA intercalator. As with the previous enediyne assays, reported assays for cleavage by these agents have all relied upon discontinuous systems and thus, molecular break lights should present an obvious advantage. FIG. 6 illustrates agent concentration dependent cleavage of break light A. Under the conditions described, this assay allows the detection of bleomycin in the nM range which represents a slight increase in sensitivity over the biochemical induction assay (BIA) and reiterates the power of this assay to detect the production of naturally-produced DNA-damaging agents.

To increase the sensitivity for the less efficient reagent Fe⁺²-EDTA, oligo concentration was increased 10-fold (32 nM), FIG. 6d. As a comparison, MPE was also examined at this higher molecular break light concentration, FIG. 6c. Finally, while ascorbate is critical for efficient DNA-cleavage by MPE and by Fe⁺²-EDTA, the addition of ascorbate did not affect DNA-cleavage by bleomycin.

Prevention of cleavage by calicheamicin—protection by CalC was assessed. Given that calicheamicin leads to double strand DNA cleavage and CalC provides calicheamicin-resistance in vivo, it was expected that the addition of CalC to an in vitro calicheamicin-induced DNA cleavage assay would inhibit DNA cleavage. To test this theory, preliminary assays were performed with supercoiled pBlusecript plasmid DNA (“pBS”) as the template, and dithiothreitol (“DTT”) as the reductive initiator. In a typical assay, purified mbp-CalC (15.0 nM) (CalC produced as a maltose binding protein-CalC fusion protein) and 30.0 nM calicheamicin were preincubated for 15 min. in a total volume of 25 μL 40 mM Tris-Cl, pH 7.5, at 37° C. Then 2.5 μL 10 mM DTT stock solution was added to the assay solution, and the assay was incubated an additional 1 hour at 37° C. DNA fragmentation was assessed by electrophoresis on a 1% agarose gel stained with ethidium bromide. Using this assay, it was found that mbp-CalC could completely inhibit calicheamicin-induced DNA cleavage at concentrations nearing 10³-fold excess of calicheamicin. Preincubation of mbp-CalC and DTT, protein removal via forced dialysis, and the subsequent use of the DTT solution as reductant did not noticeably affect the amount of DNA cleavage.

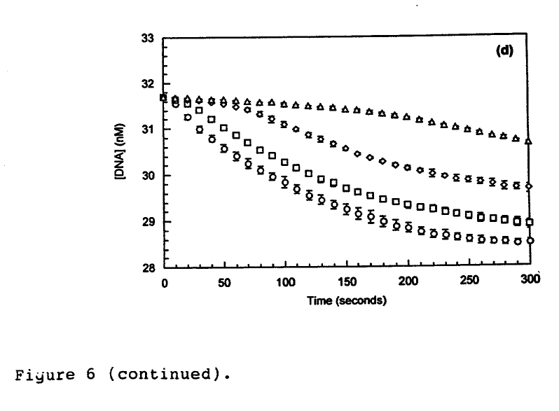

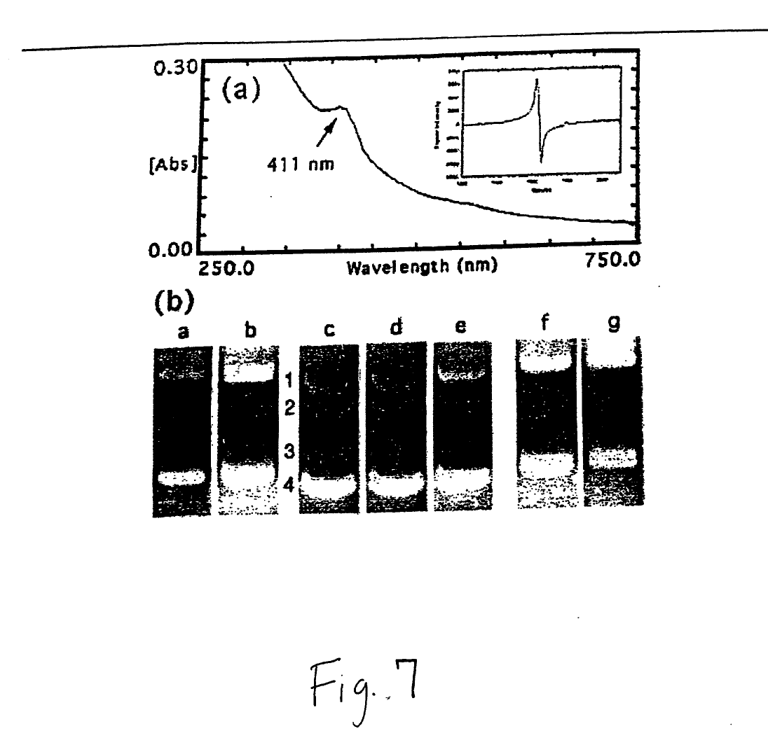

As indicated in FIG. 7, no DNA cleavage was observed in the absence of DTT or calicheamicin (lanes a and b), while efficient cleavage was demonstrated in the presence of DTT and calicheamicin (lane c). As expected, the addition of mbp-CalC completely inhibited calicheamicin-induced DNA cleavage (lane f) while the addition of mbp alone (lane d) as a control, failed to inhibit calicheamicin-induced DNA cleavage. Furthermore, preincubation of mbp-CalC with DTT (not shown), or apo-mbp-CalC (lacking the Fe cofactor)(lane e), also failed to inhibit calicheamicin-induced DNA cleavage. However, the addition of Fe⁺² or Fe⁺³ to the apo-mbp-CalC assay could reconstitute CalC activity (lane g). Reconstitution of apo-mbp-CalC was accomplished by preincubation with 1 mM FeSO₄ (Fe⁺²) or FeCl₃ (Fe⁺³) prior to the activity assay as previously described.

CalC inhibition of calicheamicin mediated DNA cleavage was examined. Two molecular break lights for the experiments are shown in FIG. 2. Break light A was comprised of a 10-base pair stem which contained the known calicheamicin recognition sequence 5′-TCCT-3′, while break light B carried the BamHI endonuclease recognition sequence 5′-GGATCC-3′. The length of break light B also considered the requirement of a 3 base pair overhang required for BamHI recognition and the stem of break light A was adjusted to a comparable length and melting temperature. The loop of both probes consisted of a T₄ loop to ensure non-hybridizing interactions. The 5′-fluorophore of both probes was fluorescein (FAM, absorbanceₘₐₓ=485 nm, emissionₘₐₓ=517 nm) while the corresponding 3′-quencher was 4-(4′-dimethylaminophenylazo)benzoic acid (DABCYL). FIG. 8 is a representation of the cleavage of break light A by calicheamicin and of break light B by BamHI.

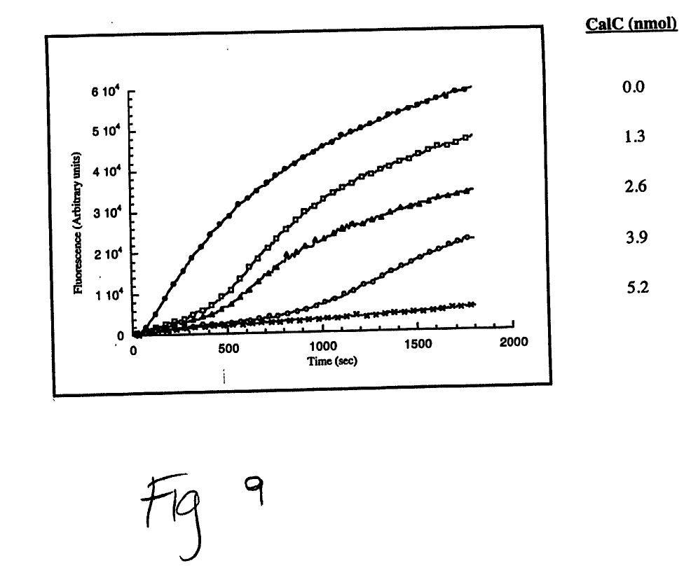

As illustrated in FIG. 9, CalC directly inhibits of calicheamicin-mediated DNA cleavage in the break light assay. 3.6 pM break light A is coincubated with 3.5 nM calicheamicin with increasing amounts of CalC. Complete inhibition of calicheamicin is achieved with roughly 2-fold excess of CalC. CalC has no effect on esperamicin-induced cleavage of DNA (data not shown).

Cleavage by the various agents investigated was compared. A direct correlation of the turnover (Vapp/[cleavage agent]) for calicheamicin, esperamicin, bleomycin, MPE, and Fe⁺²-EDTA indicates the maximum turnover when [molecular break light A (FIG. 2B)]=3.2 nM (representing at least 76.8 nM cleavage sites) occurs in the range of 0.78-1.6 nM for the enediynes, 2.5 nM for bleomycin and 125 nM for MPE. At the higher molecular break light concentration, [A]=32 nM, maximum turnover occurs in the range of 50 nM MPE and 1.3 μM Fe⁺²-EDTA. These maximum turnover values are summarized in Table 1 to correlate the cleavage efficiencies of this highly diverse group of DNA cleavage agents where MPE, assayed at both concentrations of oligonucleotide, serves as the common agent in both sets.

Table 1 suggests the addition of an intercalator (MPE) to the Fe⁺²-chelation domain enhances the cleavage efficiency almost 10³-fold in comparison to Fe⁺²-EDTA (FIG. 1E) and the addition of a specific minor groove binder bleomicin, increases this efficiency an additional 10-fold. While the cleavage efficiencies of calicheamicin and esperamicin are nearly identical, the near 10-fold enhancement over bleomycin may be attributed to direct hydrogen abstraction (versus diffusable active radical species formed from iron-dependent agents) in the formation of the DNA backbone radicals which ultimately lead to oxidative cleavage.

Significantly, Table 1 illustrates these spectacular enediynes are as efficient as an enzyme as the kcat of BamHI is identical to the observed maximum turnover of esperamicm.

Unless otherwise defined, all technical and scientific terms used herein have the same meaning as commonly understood by persons of ordinary skill in the art to which this invention belongs.

As can be appreciated from the disclosure above, the present invention has a wide variety of applications. Accordingly, the following examples are offered by way of illustration, not by way of limitation.

### EXAMPLES

Materials and Methods

Materials.

All oligonucleotides utilized for the described studies were purchased from GIBCO-BRL. Esperamicin was a generous gift of Dr. Kin Sing (Ray) Lam, Bristol-Myers Squibb and bleomycin sulfate (Blenoxane) was kindly provided by Professor Ben Shen, University of California, Davis. Wyeth-Ayerst Research Division of American Home Products provided calicheamicin. All other reagents described were obtained from commercial sources.

Spectrofluorometry.

Samples were analyzed with a FluoroMax-2 spectrofluorometer equipped with DataMax for Windows (Instruments S. A., Inc.; Edison, N.J.) and the temperature controlled (30° C., unless otherwise noted) by a Haake Circulator DC10. All samples were filtered prior to analysis and analyzed via a timebase scan (λₑₓ=485 nm, λ_{Em}=517 nm) in a Suprasil quartz cuvette (10 mm path) fitted with a magnetic stirring bar in a total volume of 2 mL. Reactions were equilibrated to the incubation temperature before initiation of DNA cleavage as was evident by a steady background emission over 10 min. Total cleavage of the labeled oligonucleotide, confirmed by polyacrylamide gel electrophoresis (PAGE), was defined as the maximum fluorescence emission possible under saturated cleaving conditions. Emission units were converted to the amount of labeled oligonucleotide used within a procedure, thereby equating labeled oligonucleotide degradation as a function of the emission of fluorescence.

### Example 1

### Design and Construction of Molecular Break Lights

Two molecular break light probes were prepared for the experiments described. FIG. 2B. Molecular break light A comprised a 10-base pair stem which contained the known calicheamicin recognition sequence 5′-TCCT-3′. See, e.g., Zein, N., et al. Science 244: 697-699 (1989). Molecular break light B carried the BamHI endonuclease recognition sequence 5′-GGATCC-3′. See, e.g., Van Dyke et al. Nuc. Acids Res. 11: 5555-5567 (1983). The design of the length of break light probe B also took into consideration the provision of a 3 base pair overhang required for BamHI recognition. The stem of break light A was adjusted to a length and melting temperature comparable to those of break light B. The loop of both probes consisted of a T₄ loop to ensure non-hybridizing interactions. Control molecules having the nucleotide sequence of A and B, but not having the fluorophore or quencher were also constructed.

The 5′-fluorophore of both probes was fluorescein (FAM, absorbanceₘₐₓ=485 nm, emissionₘₐₓ=517 nm), while the corresponding 3′-quencher was 4-(4′-dimethylaminophenylazo)benzoic acid (DABCYL). Previous studies have shown DABCYL to serve as a universal quencher in molecular beacons, and there is significant spectral overlap (1.02×10⁻¹⁵ M⁻¹ cm³) between the emission spectrum of FAM and the absorption spectrum of DABCYL. In a typical molecular beacon, the quenching efficiency of this pair via FRET has been shown to be essentially complete (99.9%), providing a significant enhancement of the signal to noise ratio as compared to typical complementary oligonucleotide pair FRET-based assays. See, e.g., Tyagi, S. et al. (1996) Nature Biotechnol. 14: 303-308 Tyagi, S., et al. (1998) Nature Biotechnol. 16: 49-53.

### Example 2

### BamHI Digestion: Enzymatic Cleavage as Proof of Principle

The cleavage of molecular break light probes A and B by BamHI was investigated. In a first assay, break light A and break light B were each incubated with 100 U BamHI. As a control, break light B was also incubated without enzyme. The incubations occurred at 37° C. in a solution containing 10 mM TrisHCl, 50 mM NaCl, 10 mM MgCl₂, and 1 mM DTT at pH 7.9.

In a second assay, break light A and break light B were each incubated with 10 U DnaseI. As a control, break light A was also incubated without enzyme. The incubations occurred at 37° C. in a solution containing 40 mM Tris HCl, 10 mM MgSO₄, and 1 mM CaCl₂ at pH 8.0.

FIG. 3A reveals a time dependent and [BamHI]-dependent increase of fluorescence only with B; A incubated with BamHI shows no change at 37° C. FIG. 3B illustrates a [DNaseI]-dependent increase of fluorescence over time both when break light A is incubated with DNase and when break light B is incubated with DNase.

In comparison, control samples containing break lights alone or break lights in the presence of BSA showed no change in fluorescence over >2 hr at 37° C.

### Example 3

### BamHI Steady State Kinetic Determination and Sensitivity Limits

Determination of BamHI (10 units/μL) specific cleavage was performed in 6 mM TrisHCl, 100 mM NaCl, 6 mM MgCl₂, and 1 mM DTT, at 37° C. and pH 7.5 with 3.2 nM of molecular break light B (FIG. 2B) and varying amounts of BamHI-specific oligonucleotide lacking the fluorophore and quenching moieties. Total substrate concentrations (including break light) were as follows: 389 nM, 196 nM, 81 nM, 42 nM, 11 nM, 7.5 nM, and 3.4 nM.

The reaction was initiated with 10 U BamHI enzyme and monitored via spectrofluorometry over a time course of fifteen minutes. The initial rate of DNA cleavage was determined from data within the first 100 seconds of initiation which was then adjusted according to equation 1. These adjusted values were utilized for the reciprocal plot from which the Michaelis-Menten kinetic parameters were determined.

V_{act}=V_{obs}([S_{act}]/[S*]) [Equation 1]

The steady-state kinetic parameters for a commercially available BamHI were determined. The dependence of BamHI hydrolysis on substrate concentration was investigated using mixtures of a fixed amount of molecular break light B and varying amounts of an analogous non-labeled oligonucleotide (lacking both FAM and DABCYL) over a wide substrate concentration range. The apparent competitive inhibition observed due to phenomenon of “carrier dilution” was corrected to give the appropriate kinetic parameters. See, e.g., Roy, et al. (1994).

As illustrated in FIG. 4a, the velocity curves decrease with an increase in initial substrate concentration, although the true velocity has actually increased, due to the carrier dilution by the non-labeled oligonucleotide. The observed velocity (Vₐₚₚ) is related to the actual velocity (V_{act}) by equation [I] where [S_{act}] and [S*] are the total substrate concentration and B concentration, respectively. The reciprocal plot after correction for this “carrier dilution” phenomenon is illustrated in FIG. 4b.

From FIG. 4b, the determined Kₘ=8.9±0.5 nM and v max=0.024±0.001 nM sec⁻¹. While these values differ slightly from previously reported values for BamHI of Kₘ (Kₘ=0.4 nM and V max=0.009 nM sec⁻¹, kinetic parameters of restriction endonucleases vary significantly depending upon the oligonucleotide substrate. Further, examination of three different commercial sources of BamHI (Promega, New England Biolabs and GIBCO BRL) gave markedly distinct specific activities (ranging roughly an order of magnitude). Thus, the differences in the reported kinetic parameters could also simply-reflect distinctions in the enzyme preparation and/or commercial assay buffers.

A recent fluorescence correlation spectroscopy (FCS) assay for the restriction endonuclease EcoRI using 0.8 nM of dual fluorophoric-labeled dsDNA and a highly specialized FCS spectrometer, reported a detection limit of 1.6 pM EcoRI. Kettling, U., et al. (1998). Under the conditions containing even slightly less oligonucleotide (0.68 nM molecular break light), cleavage was easily detectable to 3.7 pM BamHI. Furthermore, due to the significantly low signal to noise of this assay, increasing the molecular break light concentration (34 μM) lowered the detection limit into the fM range (0.12 pM BamHI). This assay was performed undert the same conditions as the other assays in this Example.

### Example 4

### Enediyne-Induced Cleavage

Molecular break light probes of the present invention were used to follow directly the extent of DNA cleavage by a specific enediyne in real time with high sensitivity. Enediyne antibiotics calicheamicin and esperamicin at varying concentrations (0.31, 0.78, 1.6, 3.2, 15.9, and 31.7 nM) were incubated in 40 mM Tris-HCl (pH 7.5) with 3.2 nM of the calicheamicin-specific labeled molecular break light oligonucleotide (A). DNA cleavage was initiated with the addition of 1 μL 100 mM dithiothreitol (“DTT”) to produce a final concentration of 50 μM DTT, and the reaction was monitored over 10 minutes via spectrofluorometry.

Two controls were used: molecular break light A was incubated with either DTT in the absence of enediyne or with enediyne in the absence of DTT.

Pseudo-first order kinetic parameters were utilized to determine the initial velocities at each given enediyne concentration. Specifically, graphical representation of the data was based upon equation 2 where [A]ₜ is the concentration of cleaved oligonucleotide at a given time (t) and [A]₀ is the initial concentration of oligonucleotide in the assay. Least squares analysis gave the slope (k), or rate, which was converted to V by the relationship in equation 3. The maximum velocity achieved (Vₘₐₓ) was then selected from the range of concentrations examined.

ln[A]ₜ=−kt+ln[A]₀ [Equation 2]

V=k[A]₀ [Equation 3]

FIG. 5A and FIG. 5B illustrate enediyne concentration dependent cleavage of break light A with either calicheamicin (FIG. 5A) or esperamicin (FIG. 5B) in the presence of excess reductive activator DTT. Under the conditions described, this assay allows the detection of calicheamicin in the pM range. No change in fluorescence was observed in the controls, incubation of molecular break light A with either DTT or enediyne alone. Furthermore, break light B was cleaved by calicheamicin at a rate identical to that of break light A.

Two distinct rates were observed in the enediyne molecular break light assay. The first (0-50 seconds) is a lag time most likely attributed to the enediyne activation while the second (50-200 seconds) is indicative to the initial velocity of DNA cleavage. To confirm this, assays were also established in which DTT and enediyne were first preincubated for 1-5 min followed by initiation via the addition of the substrate oligonucleotide. In these preincubation experiments, the previously observed “lag time” attributed to activation was no longer evident while the initial velocity of DNA cleavage was identical to that determined in the standard assay. Preincubation for longer periods (>30 min) revealed the same phenomenon.

### Example 5

### Bleomycin-Induced Cleavage

Bleomycin, an Fe⁺²-dependent nucleic acid cleavage agent, is a natural metabolite from Streptomyces verticillus. Blenoxane (a mixture containing approximately 70% bleomycin A₂ and 30% bleomycin B₂) was dissolved in water & optically standardized (ε₂₉₁=1.7×10⁴M⁻¹ cm⁻¹). Bleomycin mediated cleavage was adapted from procedures outlined by Giloni et al. J. Biol. Chem. 256: 8608-8615 (1981). Several different concentrations of bleomycin (200 nM, 100 nM, 50 nM, 25 nM, 12.5 nM, 5 nM, and 2.5 nM) were incubated in 50 mM sodium phosphate, 2.5 mM ascorbate, at pH 7.5 and 37° C. with 3.2 nM of the molecular break light A.

The reaction was initiated by the addition of 65 mM Fe(II) and monitored over 5 minutes. This protocol was repeated with the addition of 5 mM sodium ascorbate to the above conditions. Pseudo-first order kinetic parameters were utilized to determine the initial velocities at each given bleomycin concentration as previously described.

FIG. 6A illustrates agent concentration dependent cleavage of break light A by Blenoxane. Under the conditions described, this assay allows the detection of bleomycin in the nM range. Although ascorbate is critical for efficient DNA-cleavage by MPE and by Fe⁺²-EDTA, the addition of ascorbate did not affect DNA-cleavage by bleomycin.

### Example 6

### Iron (II)-Chelator-Induced Cleavage

Nucleotide cleavage by two DNA-footprinting reagents, methidiumpropyl-Fe-EDTA (MPE) (FIG. 1D) and Fe-EDTA (FIG. 1E), was evaluated using molecular break light probes. MPE and Fe⁺²-EDTA cleave DNA via the generation of diffusable hydroxy radicals which ultimately contribute to oxidative DNA cleavage.

All Fe-containing solutions were prepared fresh daily from (NH₄)₂Fe(SO₄)₂ with 1 mM H₂SO₄ to prevent hydrolysis and oxidation. EDTA-Fe(I) mediated oligonucleotide degradation was adapted from procedures outlined by Tullius et al. Meth. Enzymol. 208: 380-413 (1991).

In a first assay, 3.2 nM break light A was incubated in 40 mM Tris HCl and 2.5 mM ascorbate at 37° C. and pH 7.5. Cleavage was initiated by addition of MPE/Fe(II) in a 1.2:1 molar ratio to various concentrations. Final Fe(II) concentrations were 8 μM, 4 μM, 2 μM, 1 μM, 500 nM, 250 nM, and 125 nM. Results are shown in FIG. 6B.

In a second assay, 32 nM molecular break light A was incubated in 40 mM Tris and 2.5 mM sodium ascorbate at pH 7.5 and 37° C. Cleavage was initiated by addition of MPE/Fe(II) in a 1.2:1 molar ratio to various concentrations. Final Fe(II) concentrations were 50 nM, 125 nM, 250 nM, 500 nM, 1 μM, and 2 μM. Results are shown in FIG. 6C.

In a third assay, 32 nM break light A (calicheamicin-specific molecular break light oligonucleotide) was incubated in 40 mM Tris and 2.5 mM sodium ascorbate at pH 7.5 and 37° C. Cleavage was initiated by addition of EDTA/Fe(II) in a 2:1 molar ratio to various concentrations. Final Fe(II) concentrations were 12.5 μM, 6.3 μM, 3.1 μM, and 1.3 μM. Results are shown in FIG. 6D.

MPE-Fe(II) mediated degradation was adapted from procedures outlined by Van Dyke and Dervan. Nuc. Acids Res. 11: 5555-5567 (1983).

Pseudo-first order kinetic parameters were utilized to determine the initial velocities at each given agent concentration as previously described.

FIG. 6 illustrates agent concentration dependent cleavage of break light A. To increase the sensitivity for the less efficient reagent Fe⁺²-EDTA, oligo concentration was increased 10-fold (32 nM), (FIG. 6D). As a comparison, MPE was also examined at this higher molecular break light concentration, (FIG. 6C).

### Example 7

### Prevention of Cleavage by Calicheamicin—Protection of Supercoiled Plasmid DNA by CalC

CalC, which is found within the calicheamicin gene cluster, is known to protect DNA from degradation by calicheamicin. CalC was produced as described in the published PCT patent application WO/00/37608, entitled “Micromonospora echinospora genes encoding for biosynthesis of calicheamicin and self-resistance thereto.”

FIG. 7A is a graph of the UV-visible absorption spectra of purified mbp-CalC. The purified mpb-CalC was analyzed in the following solution: 52 μM mpb-CalC; 10 mM Tris-HCl, pH 7.5). The inset shows the results of low temperature (4.3 K) the X-band EPR analysis of CalC. 250 μM mpb-CalC containing 0.5 mol Fe per mol CalC was analyzed in 10 mM Tris-HCl, pH 7.5. The spectrometer settings were as follows: field set=2050 G; scan range=4,000G; time constant=82 s; modulation amplitude=16 G; microwave power=31 μW; frequency=9.71 Ghz; gain=1000; determined spin quantitation=90±10 μM Fe.

Given that calicheamicin causes double strand DNA cleavage and that CalC provides calicheamicin-resistance in vivo, it was expected that the addition of CalC to an in vitro calicheamicin-induced DNA cleavage assay would inhibit DNA cleavage.

To test this theory, assays were performed with supercoiled pblusecript plasmid DNA (“pBS”) as the template, and dithiothreitol (“DTT”) as the reductive initiator. In a typical assay, purified 15.0 nM mbp-CalC (CalC produced as a maltose binding protein-CalC fusion protein) and 30.0 nM calicheamicin were preincubated for 15 minutes. in a total volume of 25 μL 40 mM Tris-Cl, pH 7.5, at 37° C. 2.5 μL 10 mM DTT stock solution was added to the assay solution, and the assay was incubated an additional 1 hour at 37° C.

DNA fragmentation was assessed by electrophoresis on a 1% agarose gel stained with ethidium bromide. Using this assay, it was found that mbp-CalC could completely inhibit calicheamicin-induced DNA cleavage at concentrations nearing 10³-fold excess of calicheamicin. Preincubation of mbp-CalC and DTT, protein removal via forced dialysis, and the subsequent use of the DTT solution as reductant did not noticeably affect the amount of DNA cleavage.

As indicated in FIG. 7B, no DNA cleavage was observed in the absence of DTT or calicheamicin (lanes a and b), while efficient cleavage was demonstrated in the presence of DTT and calicheamicin (lane c). As expected, the addition of mbp-CalC completely inhibited calicheamicin-induced DNA cleavage (lane f) while the addition of mbp alone (lane d) as a control, failed to inhibit calicheamicin-induced DNA cleavage. Furthermore, preincubation of mbp-CalC with DTT (not shown), or apo-mbp-CalC (lacking the Fe cofactor)(lane e), also failed to inhibit calicheamicin-induced DNA cleavage. However, the addition of Fe⁺² or Fe⁺³ to the apo-mbp-CalC assay could reconstitute CalC activity (lane g). Reconstitution of apo-mbp-CalC was accomplished by preincubation with 1 mM FeSO₄ (Fe⁺²) or FeCl₃ (Fe⁺³) prior to the activity assay as previously described.

### Example 8

### Prevention of Cleavage by Calicheamicin—Protection of Supercoiled Plasmid DNA by CalC

Molecular break light probe A was used to assay CalC inhibition of nucleotide cleavage by calicheamicin. As illustrated in FIG. 9, CalC directly inhibits calicheamicin-mediated DNA cleavage in the break light assay.

3.6 pM break light A was coincubated with 3.5 nM calicheamicin with increasing amounts of CalC (0.0 nm, 1.3 nm, 2.6 nm, 3.9 nm, 5.2 nm).

As is shown in FIG. 9, Titration of increasing amounts of CalC into the molecular break light assay in the presence of calicheamicin completely abolishes the cleavage and, thus, the fluorescent signal. Complete inhibition of calicheamicin was achieved with roughly 2-fold excess of CalC. CalC has no effect on esperamicin-induced cleavage of DNA (data not shown).

TABLE 1
A comparison of cleavage efficiencies.

| | Agent | Vₘₐₓ (nM sec⁻¹) | Turnover (sec⁻¹)^{a} | Comparison to EDTA^{b} |
|---|---|---|---|---|
| enzymatic | BamHI | 0.024 ± 0.001 | 0.007^{c} | 4.8 × 10⁵ |
| | Esperamicin A₁ | 0.007 ± 0.001^{d} | 0.009 | 6.1 × 10⁵ |
| | Calicheamicin_{γ1}^{l} | 0.011 ± 0.002^{d} | 0.007 | 4.8 × 10⁵ |
| small | Bleomycin | 0.009 ± 0.001^{d} | 0.001 | 6.8 × 10⁴ |
| molecule | Methidiumpropyl-EDTA | 0.003 ± 0.001^{d} | 2.4 × 10⁻⁵ | 1.6 × 10³ |
| catalyzed | Methidiumpropyl-EDTA | 0.118 ± 0.004^{e} | 0.002 | 1.6 × 10³ |
| | EDTA | 0.002 ± 0.001^{e} | 1.5 × 10⁻⁶ | 1.0 |

^{a}defined as Vₘₐₓ/[Agent]; ^{b}fold enhancement over EDTA turnover; ^{c}also known as k_{cat}; ^{d}[DNA]ₜₒₜₐₗ = 3.2 nM; ^{e}[DNA]ₜₒₜₐₗ = 32 nM

What is claimed is:

1. A process for evaluating activity of a nucleic acid cleavage agent present in a sample, the process comprising:
a. incubating the sample with a probe, the probe comprising:
i. an oligonucleotide that forms a stem loop structure
ii. a fluorophore, and
iii. a quencher, wherein the fluorophore and the quencher are positioned such that the fluorophore fluoresces less when the probe is intact than when the probe is cleaved;
b. measuring the level of fluorescence of the probe; and
c. correlating amount of fluorescence with activity of the nucleic acid cleavage agent.

2. The process of claim 1, wherein the nucleic acid cleavage agent is an enzyme.

3. The process of claim 2, wherein the enzyme is a nuclease.

4. The process of claim 3, wherein the nuclease is an exonuclease.

5. The process of claim 3, wherein the nuclease is an endonuclease.

6. The process of claim A5, wherein the enzyme is a restriction endonuclease.

7. The process of claim 1, wherein the nucleic acid cleavage agent is a small molecule.

8. The process of claim 1, wherein the nucleic acid cleavage agent is an enediyne.

9. The process of claim 1, wherein the nucleic acid cleavage agent cleaves the probe in the single stranded portion of the stem loop structure.

10. The process of claim 1, wherein the nucleic acid cleavage agent cleaves the probe in the double stranded portion of the stem loop structure.

11. The process of claim 1 wherein the fluorophore and quencher are internally coupled to the probe.

12. The process of claim 1 wherein the fluorophore and quencher are coupled to the 5′ and/or 3′ ends of the probe.

13. The process of claim 1 wherein the nucleic acid cleavage agent cleaves the probe at a site between the quencher and the fluorophore.

14. The process of claim 1 wherein the probe is immobilized to a solid surface.

15. A process for detecting the presence of a nucleic acid cleavage agent in a sample, the process comprising:
a. incubating the sample with a probe, the probe comprising:
i. an oligonucleotide that forms a stem loop structure
ii. a fluorophore, and
iii. a quencher, wherein the fluorophore and the quencher are positioned such that the fluorophore fluoresces less when the probe is intact than when the probe is cleaved; and
b. measuring the level of fluorescence of the probe.

16. The process of claim 15, wherein the nucleic acid cleavage agent is an enzyme.

17. The process of claim 16, wherein the enzyme is a nuclease.

18. The process of claim 17, wherein the nuclease is an exonuclease.

19. The process of claim 17, wherein the nuclease is an endonuclease.

20. The process of claim 19, wherein the enzyme is a restriction endonuclease.

21. The process of claim 15, wherein the nucleic acid cleavage agent is a small molecule.

22. The process of claim 15, wherein the nucleic acid cleavage agent is an enediyne.

23. The process of claim 15, wherein the nucleic acid cleavage agent cleaves the probe in the single stranded portion of the stem loop structure.

24. The process of claim 15, wherein the nucleic acid cleavage agent cleaves the probe in the double stranded portion of the stem loop structure.

25. The process of claim 15 wherein the fluorophore and quencher are internally coupled to the probe.

26. The process of claim 15 wherein the fluorophore and quencher are coupled to the 5′ and/or 3′ ends of the probe.

27. The process of claim 15 wherein the nucleic acid cleavage agent cleaves the probe at a site between the quencher and the fluorophore.

28. The process of claim 15 wherein the probe is immobilized to a solid surface.

29. A process for evaluating activity of a nucleic acid cleavage agent present in a sample, the process comprising:
a. incubating the sample with a probe, the probe comprising:
i. an oligonucleotide that forms a stem loop structure and comprises a recognition site for the nucleotide cleavage agent;
ii. a fluorophore, and
iii. a quencher, wherein the fluorophore and the quencher are positioned such that the fluorophore fluoresces less when the probe is intact than when the probe is cleaved;
b. measuring the level of fluorescence of the probe; and
c. correlating amount of fluorescence with activity of the nucleic acid cleavage agent.

30. The process of claim 29, wherein the nucleic acid cleavage agent is an enzyme.

31. The process of claim 30, wherein the enzyme is a nuclease.

32. The process of claim 31, wherein the nuclease is an exonuclease.

33. The process of claim 31, wherein the nuclease is an endonuclease.

34. The process of claim 33, wherein the enzyme is a restriction endonuclease.

35. The process of claim 29, wherein the nucleic acid cleavage agent is a small molecule.

36. The process of claim 29, wherein the nucleic acid cleavage agent is an enediyne.

37. The process of claim 29, wherein the recognition site is located in the single stranded portion of the stem loop structure.

38. The process of claim 29, wherein the recognition site is located in the double stranded portion of the stem loop structure.

39. The process of claim 29, wherein the recognition site spans the junction between the single stranded and the double stranded portions of the stem loop structure.

40. The process of claim 29 wherein the fluorophore and quencher are internally coupled to the probe.

41. The process of claim 29 wherein the fluorophore and quencher are coupled to the 5′ and/or 3′ ends of the probe.

42. The process of claim 29 wherein the recognition site is located at a site between the quencher and the fluorophore.

43. The process of claim 29 wherein the probe is immobilized to a solid surface.

44. A process for detecting the presence of a nucleic acid cleavage agent in a sample, the process comprising:
a. incubating the sample with a probe, the probe comprising:
i. an oligonucleotide that forms a stem loop structure and comprises a recognition site for the nucleotide cleavage agent;
ii. a fluorophore, and
iii. a quencher, wherein the fluorophore and the quencher are positioned such that the fluorophore fluoresces less when the probe is intact than when the probe is cleaved; and
b. measuring the level of fluorescence of the probe.

45. The process of claim 44, wherein the nucleic acid cleavage agent is an enzyme.

46. The process of claim 45, wherein the enzyme is a nuclease.

47. The process of claim 46, wherein the nuclease is an exonuclease.

48. The process of claim 46, wherein the nuclease is an endonuclease.

49. The process of claim 48, wherein the enzyme is a restriction endonuclease.

50. The process of claim 44, wherein the nucleic acid cleavage agent is a small molecule.

51. The process of claim 44, wherein the nucleic acid cleavage agent is an enediyne.

52. The process of claim 44, wherein the recognition site is located in the single stranded portion of the stem loop structure.

53. The process of claim 44, wherein the recognition site is located in the double stranded portion of the stem loop structure.

54. The process of claim 44, wherein the recognition site spans the junction between the single stranded and the double stranded portions of the stem loop structure.

55. The process of claim 44 wherein the fluorophore and quencher are internally coupled to the probe.

56. The process of claim 44 wherein the fluorophore and quencher are coupled to the 5′ and/or 3′ ends of the probe.

57. The process of claim 44 wherein the recognition site is located at a site between the quencher and the fluorophore.

58. The process of claim 44 wherein the probe is immobilized to a solid surface.

59. A process for evaluating activity of a nucleic acid cleavage agent, the process comprising:
a. incubating the nucleotide cleavage agent with a first probe, the first probe comprising:
i. an oligonucleotide that forms a stem loop structure and having a first sequence;
ii. a fluorophore, and
iii. a quencher, wherein the fluorophore and the quencher are positioned such that the fluorophore fluoresces less when the probe is intact than when the probe is cleaved;
b. measuring level of the fluorescence of the first probe;
c. incubating the nucleotide cleavage agent with a second probe, the second probe comprising:
i. an oligonucleotide that forms a stem loop structure and having a second sequence;
ii. a fluorophore, and
iii. a quencher, wherein the fluorophore and the quencher are positioned such that the fluorophore does not fluoresce when the probe is intact and does fluoresce when the probe is cleaved;
d. measuring level of the fluorescence of the second probe;
e. comparing the level of fluorescence of the first probe to the level of fluorescence of the second probe; and
f. correlating the amount of fluorescence of the first and second probes with activity of the nucleic acid cleavage agent.

60. The process of claim 59, wherein steps (a) and (c) are carried out in separate reaction vessels.

61. The process of claim 59, wherein steps (a) and (c) are carried out in the same reaction vessel.

62. The process of claim 61, wherein the first probe comprises a first fluorophore and the second probe comprises a second fluorophore, and wherein the first and second fluorophores are distinguishable from one another.

63. The process of claim 59, wherein the nucleic acid cleavage agent is an enzyme.

64. The process of claim 63, wherein the enzyme is a nuclease.

65. The process of claim 64, wherein the nuclease is an exonuclease.

66. The process of claim 64, wherein the nuclease is an endonuclease.

67. The process of claim 66, wherein the enzyme is a restriction endonuclease.

68. The process of claim 59, wherein the nucleic acid cleavage agent is a small molecule.

69. The process of claim 59, wherein the nucleic acid cleavage agent is an enediyne.

70. The process of claim 59, wherein the nucleic acid cleavage agent cleaves the probe in the single stranded portion of the stem loop structure.

71. The process of claim 59, wherein the nucleic acid cleavage agent cleaves the probe in the double stranded portion of the stem loop structure.

72. A process for evaluating activity of a nucleic acid cleavage agent, the process comprising:
a. incubating the nucleotide cleavage agent with a probe in a first set of conditions, the probe comprising:
i. an oligonucleotide that forms a stem loop structure;
ii. a fluorophore, and
iii. a quencher, wherein the fluorophore and the quencher are positioned such that the fluorophore fluoresces less when the probe is intact than when the probe is cleaved;
b. measuring level of fluorescence of the probe in the first set of conditions;
c. incubating the nucleotide cleavage agent with the probe in a second set of conditions;
d. measuring level of fluorescence of the probe in the second set of conditions;
e. comparing the level of fluorescence of the probe in the first set of conditions to the level of fluorescence of the probe in the second set of conditions; and
f. correlating the level of fluorescence in the first and second conditions to the activity of the nucleic acid cleavage agent.

73. The process of claim 72, wherein the nucleic acid cleavage agent is an enzyme.

74. The process of claim 73, wherein the enzyme is a nuclease.

75. The process of claim 74, wherein the nuclease is an exonuclease.

76. The process of claim 74, wherein the nuclease is an endonuclease.

77. The process of claim 76, wherein the enzyme is a restriction endonuclease.

78. The process of claim 72, wherein the nucleic acid cleavage agent is a small molecule.

79. The process of claim 72, wherein the nucleic acid cleavage agent is an enediyne.

80. The process of claim 72, wherein the nucleic acid cleavage agent cleaves the probe in the single stranded portion of the stem loop structure.

81. The process of claim 72, wherein the nucleic acid cleavage agent cleaves the probe in the double stranded portion of the stem loop structure.

82. A process for evaluating the effectiveness of a nucleotide protective agent, the process comprising:
a. incubating a nucleotide cleavage agent and a probe, the probe comprising:
i. an oligonucleotide that forms a stem loop structure
ii. a fluorophore, and
iii. a quencher, wherein the fluorophore and the quencher are positioned such that the fluorophore fluoresces less when the probe is intact than when the probe is cleaved;
b. measuring the level of fluorescence of the probe as incubated in step (a);
c. incubating the nucleotide protective agent, the nucleotide cleavage agent, and the probe;
d. measuring the level of fluorescence of the probe as incubated in step (c);
e. comparing the levels of fluorescence measured in steps (b) and (d); and
f. correlating amount of difference in the fluorescence levels measured in steps (b) and (d) with the effectiveness of the nucleotide protective agent.

83. The process of claim 82, wherein the nucleic acid cleavage agent is an enzyme.

84. The process of claim 83, wherein the enzyme is a nuclease.

85. The process of claim 84, wherein the nuclease is an exonuclease.

86. The process of claim 84, wherein the nuclease is an endonuclease.

87. The process of claim 86, wherein the enzyme is a restriction endonuclease.

88. The process of claim 82, wherein the nucleic acid cleavage agent is a small molecule.

89. The process of claim 82, wherein the nucleic acid cleavage agent is an enediyne.

90. The process of claim 82, wherein the nucleic acid cleavage agent cleaves the probe in the single stranded portion of the stem loop structure.

91. The process of claim 82, wherein the nucleic acid cleavage agent cleaves the probe in the double stranded portion of the stem loop structure.

92. An oligonucleotide probe comprising:
a. an oligonucleotide that forms a stem loop structure and comprises a recognition site for a nucleotide cleavage agent;
b. a fluorophore, and
c. a quencher, wherein the fluorophore and the quencher are positioned such that the fluorophore fluoresces less when the probe is intact than when the probe is cleaved.

93. The probe of claim 92, wherein the nucleic acid cleavage agent is an enzyme.

94. The probe of claim 93, wher ein the enzyme is a nuclease.

95. The probe of claim 94, wherein the nuclease is an exonuclease.

96. The probe of claim 94, wherein the nuclease is an endonuclease.

97. The probe of claim 96, wherein the enzyme is a restriction endonuclease.

98. The probe of claim 92, wherein the nucleic acid cleavage agent is a small molecule.

99. The probe of claim 92, wherein the nucleic acid cleavage agent is an enediyne.

100. A kit comprising at least one probe according to claim 92.

101. The kit of claim 100, further comprising at least one nucleotide cleavage agent that recognizes the recognition site.

102. The probe of claim 92, wherein the nucleic acid cleavage agent cleaves the probe in the single stranded portion of the stem loop structure.

103. The probe of claim 92, wherein the nucleic acid cleavage agent cleaves the probe in the double stranded portion of the stem loop structure.
